(19) United States
(12) Patent Application Publication
(10) Pub. No.: US 2007/0003585 A1
(43) Pub. Date: Jan. 4, 2007

(54) Topical skin treating compositions

(75) Inventors: Kathleen L. Clark, Medusa, NY (US); Jeffrey S. Reynolds, Greenville, NY (US)

Correspondence Address: NATH & ASSOCIATES, PLLC, 112 South West Street, Alexandria, VA, 22314, US

(73) Assignee: STIEFEL LABORATORIES, INC., Coral Gables, FL (US)

(21) Appl. No.: 11/476,558

(22) Filed: Jun. 29, 2006

Related U.S. Application Data
(60) Provisional application No. 60/694,799, filed on Jun. 29, 2005.

(51) Int. Cl. A61K 31/36; A61K 31/203; A61K 31/192

(52) U.S. Cl. 424/401; 514/559; 514/568

(57) ABSTRACT

Topical compositions and methods of using same for treating various skin disorders or conditions. In a particular aspect, these compositions comprise a storage-stable mixture of a benzoyl peroxide-containing composition, an antibiotic or a pharmaceutically acceptable salt or ester thereof, and a retinoid or a pharmaceutically acceptable salt thereof. In an alternative embodiment, these compositions comprise a storage-stable mixture of a retinoid or a pharmaceutically acceptable salt thereof and either a benzoyl peroxide-containing composition or an antibiotic or a pharmaceutically acceptable salt or ester thereof.

This application claims priority to U.S. Provisional Patent Application Ser. No. 60/694,799, filed on Jun. 29, 2005, the contents of which are hereby incorporated by reference in their entirety.

### FIELD OF THE INVENTION

The present subject matter relates generally to topical compositions and methods of using same for treating various skin disorders or conditions. In a particular aspect, these compositions comprise a storage-stable mixture of a benzoyl peroxide-containing composition, an antibiotic or a pharmaceutically acceptable salt or ester thereof, and a retinoid or a pharmaceutically acceptable salt thereof. In an alternative embodiment, these compositions comprise a storage-stable mixture of a retinoid or a pharmaceutically acceptable salt thereof and either a benzoyl peroxide-containing composition or an antibiotic or a pharmaceutically acceptable salt or ester thereof.

### BACKGROUND OF THE INVENTION

Skin disorders involving the sebaceous glands and follicles in humans include conditions such as acne and rosacea, as well as other noninfectious dermatological diseases involving microorganisms. Such disorders are often marked by inflammation.

Acne is a common skin disorder characterized by blackheads, whiteheads, papules, pustules, cysts, and various sized nodules and scars which, in the inflammatory state of the disorder, are contaminated with bacteria such as Propionibacterium acnes. The disorder affects skin areas where the sebaceous glands are most active, and bacterial infection can occur in the sebaceous follicles.

In the past, these dermatological disorders have been treated with oral and/or topical antibacterial agents. The oral antibiotics used include tetracycline, erythromycin, and minocycline. The topical compositions used have separately contained the antibiotics tetracycline, erythromycin, and clindamycin; retinoids such as retinoic acid or tretinoin; and benzoyl peroxide, which exerts its antibacterial action via its potent oxidizing properties. However, the strong oxidizing properties of a peroxide can often result in unstable compositions. Benzoyl peroxide also can act as a sebosuppressant, an irritant, and a comedolytic agent.

One currently available product, Cleocin T® brand clindamycin topical solution by Pharmacia & Upjohn Company of Kalamazoo, Mich., is a topical solution containing 1% of clindamycin. Cleocin T®, however, has several drawbacks. For one, the formulation contains 50% isopropyl alcohol and water. As such, this formulation often proves to be excessively drying and irritating to the skin. Second, the composition as dispensed by the pharmacist lacks the stability necessary for extended storage at room temperature.

Topical compositions combining at least two active antibacterial agents have been proposed as a treatment to these disorders. These compositions typically require compounding by the pharmacist and must be refrigerated. After three months of refrigeration, the compositions lose potency and effectiveness and must be replaced with a new batch.

For example, one currently available combination product is Benzamycin® brand topical gel (Dermik Laboratories, Berwyn, Pa.) which contains 3% of erythromycin and 5% of benzoyl peroxide. Benzamycin®, however, has several drawbacks. First, the product is supplied to pharmacies as a benzoyl peroxide gel in a first container and erythromycin powder in a second container. The product thus requires compounding by the pharmacist, who must (1) dissolve the erythromycin in alcohol, (2) add the erythromycin solution to the gel, and (3) stir until homogeneous in appearance. Second, the alcohol present in the composition as dispensed amounts to 16% of the total composition, which has proven to be excessively drying and irritating to the skin, particularly in combination with the benzoyl peroxide. Third, the composition as dispensed by the pharmacist (i.e., after reconstitution or compounding) lacks the stability necessary for extended storage at room temperature. The combination product can be stored under refrigeration for up to three (3) months.

Similarly, the currently available combination product BenzaClin® is a topical gel containing 1% of clindamycin and 5% of benzoyl peroxide. BenzaClin®, however, also has several drawbacks. For example, the product lacks the stability necessary for extended storage at room temperature since the combined product can only be stored for up to three (3) months. Accordingly, it must be compounded by a pharmacist since it is supplied to pharmacies as a benzoyl peroxide gel in a first container and clindamycin powder in a second container. By requiring compounding by pharmacists, the product also has variability/impurity problems, which are the result of the drug forming partially dissolved or undissolved aggregates. This causes some patients to report that the product sometimes feels “gritty” when applied to the skin, further exacerbating the inflammation and irritation problem due to skin abrasion. Lastly, this composition must be topically applied at least twice a day to be effective in accordance with label directions.

Another known topical composition for the treatment of acne is described in U.S. Pat. No. 6,117,843. This patent describes topical therapeutic compositions for the treatment of acne containing a combination of benzoyl peroxide and clindamycin. The clindamycin used in the disclosed compositions has a pre-combination pH of 5.9 to 6.9. Additionally, the disclosed compositions must be administered twice a day to be effective for the treatment of acne.

These presently known compositions for the treatment of acne are formulated for administration to patients twice per day and it has been reported that patient compliance with compositions that must be administered twice per day tends to be irregular, especially among teenagers who are the primary sufferers of acne.

Lastly, these current treatment options pose a significant risk of adverse side effects. Clindamycin, which is well absorbed through the skin, has been associated with colitis, diarrhea, and bloody diarrhea. Severe colitis may result in death. Likewise, benzoyl peroxide is a known skin-irritant that may not be well received by the skin. Similarly, retinoids also commonly are irritating, particularly to people with sensitive skin. Accordingly, there is a need to reduce the potential side effects of these prior compositions by reducing the number of required daily exposures to them.

As would be expected, compositions containing a combination of three of these active ingredients have proven to be even more difficult to manufacture than those compositions containing two active ingredients. Despite the inherent advantages to such triple active combinations, the difficulties in formulating a stable product have so far prevented the development of any products containing all three of an antibiotic, a retinoid, and benzoyl peroxide. Similarly, there have been many difficulties in formulating products containing a retinoid and at least one of benzoyl peroxide and an antibiotic. Accordingly, there remains a need for such products for the treatment of various skin disorders, such as acne.

Other efforts at improving the stability of combination products in particular have relied on the use of novel packaging that keeps the active agents separated to maintain stability until the time of use. However, compounding is still necessary at the time of dispensing, and stability remains a problem because the product must be used immediately upon being prepared.

For these reasons, it would be desirable to provide improved compositions and methods for formulating compositions for the treatment of acne. In particular, it would be desirable to provide products combining the activity of an antibiotic compound, such as clindamycin, with the activity of benzoyl peroxide and with the activity of a retinoid, with few or none of the disadvantages described above. Such compositions should overcome the formulation and stability problems which have been associated with the prior compositions, and provide improved compositions which are less irritating, easy to formulate, have a smooth consistency after formulation, are adequately stable, and have a sufficiently long storage life with or without refrigeration.

Similarly, it would likewise be desirable to provide products combining the activity of a retinoid with the activity of either an antibiotic compound, such as clindamycin, or with the activity of benzoyl peroxide, with few or none of the disadvantages described above. Such compositions should provide improved compositions which are less irritating, easy to formulate, have a smooth consistency after formulation, are adequately stable, and have a sufficiently long storage life with or without refrigeration.

Accordingly, there remains a need for an effective topical composition for the treatment of skin disorders that is storage-stable for an extended period of time, easy to formulate, and substantially uniform.

### SUMMARY OF THE INVENTION

The present subject matter relates generally to topical compositions useful in treating various skin disorders or conditions.

In a preferred embodiment, the present subject matter relates to a topical composition for treating a skin disorder or condition, which comprises:

a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,

wherein the composition has a final pH of about 3 to about 8, and wherein the composition has a viscosity lower than the viscosity of the benzoyl peroxide-containing composition before obtaining said storage-stable mixture.

In a further preferred embodiment, the present subject matter also relates to a topical composition for treating a skin disorder or condition, which comprises:

a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,

wherein one or more of said benzoyl peroxide, said antibiotic or a pharmaceutically acceptable salt thereof, and said retinoid or a pharmaceutically acceptable salt thereof is encapsulated or entrapped in a solid or a semi-solid ingredient, and

wherein the composition has a final pH of about 3 to about 8.

In another preferred embodiment, the present subject matter relates to a topical composition for treating a skin disorder or condition, which comprises:

a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,

wherein said composition maintains a concentration of each of said benzoyl peroxide, said antibiotic or a pharmaceutically acceptable salt or ester thereof, and said retinoid or a pharmaceutically acceptable salt thereof ingredients that is at least 90% of a label claim for each of said ingredients.

In still another preferred embodiment, the present subject matter also relates to a method for treating a skin disorder or condition in a patient comprising topically administering to a patient in need thereof a topical composition in an amount effective to treat said skin disorder, wherein said composition comprises:

a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,

wherein the composition has a final pH of about 3 to about 8, and wherein the composition has a viscosity lower than the viscosity of the benzoyl peroxide-containing composition before obtaining said storage-stable mixture.

In a further preferred embodiment, the present subject matter relates to a topical composition for treating a skin disorder or condition, which comprises:

a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,

wherein the composition has a final pH of about 3 to about 8 that contributes to stability of said topical composition.

In yet another preferred embodiment, the present subject matter relates to a topical composition for treating a skin disorder or condition, which comprises:

a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,

wherein the composition is storage stable at a refrigerated temperature of not more than 15° C. for at least 60 days.

In a further preferred embodiment, the present subject matter also relates to a topical composition for treating a skin disorder or condition, which comprises:

a storage-stable mixture of 1) a retinoid or a pharmaceutically acceptable salt thereof, 2) either a benzoyl peroxide-containing composition or an antibiotic or a pharmaceutically acceptable salt or ester thereof, and 3) a pharmaceutically acceptable carrier,

wherein one or more of said retinoid or a pharmaceutically acceptable salt thereof, said benzoyl peroxide, and said antibiotic or a pharmaceutically acceptable salt thereof is encapsulated or entrapped in a solid or a semi-solid ingredient, and

wherein the composition has a final pH of about 3 to about 8.

In another preferred embodiment, the present subject matter relates to a topical composition for treating a skin disorder or condition, which comprises:

a storage-stable mixture of 1) a retinoid or a pharmaceutically acceptable salt thereof, 2) either a benzoyl peroxide-containing composition or an antibiotic or a pharmaceutically acceptable salt or ester thereof, and 3) a pharmaceutically acceptable carrier,

wherein said composition maintains a concentration of each of said retinoid or a pharmaceutically acceptable salt thereof and said benzoyl peroxide or said antibiotic or a pharmaceutically acceptable salt or ester thereof ingredients that is at least 90% of a label claim for each of said ingredients.

In a further preferred embodiment, the present subject matter relates to a topical composition for treating a skin disorder or condition, which comprises:

a storage-stable mixture of 1) a retinoid or a pharmaceutically acceptable salt thereof, 2) either a benzoyl peroxide-containing composition or an antibiotic or a pharmaceutically acceptable salt or ester thereof, and 3) a pharmaceutically acceptable carrier,

wherein the composition has a final pH of about 3 to about 8 that contributes to stability of said topical composition.

In yet another preferred embodiment, the present subject matter relates to a topical composition for treating a skin disorder or condition, which comprises:

a storage-stable mixture of 1) a retinoid or a pharmaceutically acceptable salt thereof, 2) either a benzoyl peroxide-containing composition or an antibiotic or a pharmaceutically acceptable salt or ester thereof, and 3) a pharmaceutically acceptable carrier,

wherein the composition is storage stable at a refrigerated temperature of not more than 15° C. for at least 60 days.

### DETAILED DESCRIPTION OF THE INVENTION

### Definitions

As used herein, the term “acne” refers to a common inflammatory disease of the pilosebaceous glands characterized by comedones, papules, pustules, inflamed nodules, superficial pus-filled cysts, and (in extreme cases) canalizing and deep, inflamed, sometimes purulent sacs. Types of acne within the scope of the present subject matter include acne vulgaris or topical acne. “Acne” is caused by an interaction among hormones, keratin, sebum, and bacteria. One common bacterial causative agent is Propionibacterium acnes.

As used herein, the terms “administering”, “administration”, and like terms refer to any method which, in sound medical or cosmetic practice, delivers the composition to a subject in such a manner as to provide a positive effect on a dermatological disorder, condition, or appearance. The compositions are preferably administered such that they cover the entire area to be treated. “Direct administration” refers to any method which, in sound medical or cosmetic practice, delivers the composition to a subject without the use of another composition, delivery agent, or device. “Indirect administration” refers to any method which, in sound medical or cosmetic practice, delivers the composition to a subject with the use of at least one other composition, delivery agent, or device.

As used herein, the phrase “commercial purposes” refers to any purposes requiring any length of time or storage condition in accordance with FDA rules or regulations, including shipping time, storage, distribution, and refrigeration.

As used herein, the phrases an “effective amount” or a “therapeutically effective amount” of an active agent or ingredient, or pharmaceutically active agent or ingredient, which are synonymous herein, refer to an amount of the pharmaceutically active agent sufficient enough to have a positive effect on the area of application. Accordingly, these amounts are sufficient to modify the skin disorder, condition, or appearance to be treated but low enough to avoid serious side effects, within the scope of sound medical or dermatological advice. A therapeutically effective amount of the pharmaceutically active agent will cause a substantial relief of symptoms when applied repeatedly over time. Effective amounts of the pharmaceutically active agent will vary with the particular condition or conditions being treated, the severity of the condition, the duration of the treatment, the specific components of the composition being used, and like factors.

As used herein, the phrase an “extended period of time” refers to the shelf life of the presently preferred compositions, including time spent on the shelf at a pharmacy as well as the entire time period after sale of the composition during which the composition remains effective for the indicated use.

As used herein, the phrase “label claim” refers to statements made on a label or literature accompanying a pharmaceutical product for sale. In this regard, the phrase “label claim” is intended to include indications on the label, packaging, and or literature of a pharmaceutical product of the amount(s) of any active ingredient(s) present in that product.

As used herein, the term “pediatric” refers to the branch of medicine involving the treatment of children, and in particular to various specific treatments for children, or non-adult patients. In this regard, pediatric treatments are primarily intended for patients having an age of up to and including 18 years.

As used herein, the phrase “pharmaceutically acceptable salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, naturally and synthetically derived amino acids.

Non-limiting examples of base salts include ammonium salts; alkali metal salts, such as sodium and potassium salts; alkaline earth metal salts, such as calcium and magnesium salts; salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained.

As used herein, the phrases “refrigeration” or “refrigerated temperature refers to a temperature of not more than about 15° C.

As used herein, the phrase “room temperature” refers to a temperature of about 20° C. to about 25° C.

As used herein, the term “sensitivity” refers to the degree of skin irritation or skin inflammation, as exemplified by parameters in suitable assays for measuring sensitivity, inflammation, irritation, and the like. One such assay is the Jordan-King assay.

As used herein, the phrases “storage stable” or “storage-stable” are used interchangeably and refer to the ability of the present compositions to have a long shelf life, including time spent on the shelf at a pharmacy as well as the entire time period after sale of the composition, during which time the composition maintains its effectiveness and pharmaceutically acceptable appearance. Accordingly, the present compositions are stable in that they exhibit a minimum amount of degradation during an extended period of storage.

As used herein, a “treatment” or “treating” of a skin disease, disorder, or condition encompasses alleviation of at least one symptom thereof, a reduction in the severity thereof, or the delay, prevention, or inhibition of the progression thereof. Treatment need not mean that the disease, disorder, or condition is totally cured. A useful composition herein needs only to reduce the severity of a skin disease, disorder, or condition, reduce the severity of symptoms associated therewith, provide improvement to a patient's quality of life, or delay, prevent, or inhibit the onset of a skin disease, disorder, or condition.

Other terms as used herein are meant to be defined by their well-known meanings in the art.

### Topical Compositions

The subject matter expressed herein relates generally to various topical compositions for treating a skin disorder, disease, or condition, and to methods for treating such skin diseases, disorders, or conditions using the same.

In one preferred aspect in this regard, the present subject matter relates to a topical composition for treating a skin disorder or condition, which comprises a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier. In a preferred embodiment, the composition is formulated so that it has a final pH of about 3 to about 8.

In a particularly preferred embodiment of the present subject matter, the present compositions are formulated to have a viscosity lower than the viscosity of the benzoyl peroxide-containing composition before obtaining the storage-stable mixture. In an alternative particularly preferred embodiment, the present compositions are formulated so that one or more of the benzoyl peroxide, the antibiotic or a pharmaceutically acceptable salt or ester thereof, and the retinoid or a pharmaceutically acceptable salt thereof is encapsulated or entrapped in a solid or semi-solid ingredient.

In another alternative particularly preferred embodiment of the present subject matter, the present compositions are formulated to minimize the amount of degradates formed of the active ingredients present. In this regard, preferred compositions herein are preferably capable of effectively maintaining a concentration of each of the benzoyl peroxide, antibiotic or a pharmaceutically acceptable salt or ester thereof, and retinoid or a pharmaceutically acceptable salt thereof ingredients that is at least 90% of a label claim for each of these ingredients.

In a particularly preferred embodiment, the present compositions are capable of maintaining a concentration of each of the benzoyl peroxide, antibiotic or a pharmaceutically acceptable salt or ester thereof, and retinoid or a pharmaceutically acceptable salt thereof ingredients that is at least 90% of a label claim for each of these ingredients for at least 30 days either under refrigeration or at room temperature. In a further preferred embodiment, the present compositions are capable of maintaining these concentrations of the active ingredients for at least 60 days either under refrigeration or at room temperature. In another preferred embodiment, the present compositions are capable of maintaining these concentrations of the active ingredients for at least 90 days either under refrigeration or at room temperature. In still another preferred embodiment, the present compositions are capable of maintaining these concentrations of the active ingredients for at least 6 months under refrigeration.

A further alternative particularly preferred embodiment of the present subject matter relates to topical compositions containing each of these active ingredients at a final composition pH of about 3 to about 8 sufficient to contribute to product stability. Another alternative embodiment relates to combination compositions that are storage stable at a refrigerated temperature of not more than 15° C. for at least 60 days.

The benzoyl peroxide component of the present compositions is preferably introduced in an initial benzoyl peroxide-containing composition formed as a solution, dispersion, or suspension. The benzoyl peroxide is pharmaceutical grade. This benzoyl peroxide may be in the form of a slurry of a finely divided powder, or in the form of a hydrous granular material which may have its particle size reduced accordingly during processing according to the present subject matter. Preparation of suitable benzoyl peroxide constituents is well described in the medical and patent literature.

The benzoyl peroxide component of the present compositions is generally present at an amount of between about 0.1% to about 10% by weight of the total composition of benzoyl peroxide. In a preferred embodiment, the compositions contain from about 0.5% to about 5% by weight of the total composition of benzoyl peroxide. Additionally, the present compositions are unique in that they are preferably capable of effectively maintaining a level of benzoyl peroxide that is at least 90% of the label claim for the benzoyl peroxide.

According to this embodiment, the initial benzoyl peroxide-containing composition, prior to mixing, has a preferred viscosity of about 25,000 to about 1,250,000 centipoises.

In another preferred embodiment, the particle size of the benzoyl peroxide can be reduced prior to inclusion in the present compositions. Such reduction in particle size can be carried out through processing, such as processing involving a milling process or a micronization process, or the use of solvents.

The antibiotic component of the present compositions is generally present at an amount of from about 0.5% to about 3% by weight of the total composition. Additionally, the present compositions are unique in that they are preferably capable of effectively maintaining a level of antibiotic that is at least 90% of the label claim for the clindamycin.

Any of a wide variety of antibiotics known as useful in treating skin diseases, disorders, or conditions is contemplated as capable of being included in the present compositions. In this regard, preferred non-limiting examples of antibiotics useful in the present compositions include clindamycin, tetracycline, erythromycin, lincomycin, azithromycin, carbomycin, chlortetracycline, clarithromycin, demeclocycline, doxycycline, gentamicin, josamycin, kanamycin, leucomycins, meomycin, methacycline, midecamycins, miokamycin, oleandomycin, oxytetracycline, primycin, ribostamycin, rokitamycin, rolitetracycline, rosaramicin, roxithromycin, spectinomycin, spiramycin, streptomycin, sulfacetamide, sulfabenzamide, sulfadiazine, sulfadoxine, sulfamerazine, sulfamethazine, sulfamethizole, sulfamethoxazole, tobramycin, troleandomycin, salts thereof, esters thereof, derivatives thereof, and mixtures thereof. Clindamycin, erythromycin, and tetracycline, as well as salts or derivatives thereof, are especially preferred in this regard. In a most preferred embodiment, the antibiotic is clindamycin or a salt or derivative thereof.

In this regard, the antibiotic component of the present compositions is preferably a pharmaceutical grade salt or ester of clindamycin. Pharmaceutically acceptable salts or esters of clindamycin refer to those which possess the desired pharmacological activity and which are neither biologically nor otherwise undesirable. Clindamycin phosphate (ester) and clindamycin hydrochloride (salt) are preferred pharmaceutically acceptable salts and esters of clindamycin which can be used in the present compositions due to their compatibility with gelling agents and extensive history of topical use.

The retinoid component of the present compositions is preferably a pharmaceutical grade salt of the retinoid. Pharmaceutically acceptable salts, esters, or derivatives of retinoids refer to those which possess the desired pharmacological activity and which are neither biologically nor otherwise undesirable. The retinoid component of the present compositions is generally present at an amount of from about 0.01% to about 1.5% by weight of the total composition. In a particularly preferred embodiment, the retinoid is present at an amount of about 0.01% to about 0.5% by weight of the total composition.

Additionally, the present compositions are unique in that they are preferably capable of effectively maintaining a level of retinoid that is at least 90% of the label claim for the retinoid.

In another preferred embodiment, the particle size of the retinoid can be reduced prior to inclusion in the present compositions. Such reduction in particle size can be carried out through processing, such as processing involving a milling process or a micronization process, or the use of solvents.

Any of a wide variety of retinoids known as useful in treating skin diseases, disorders, or conditions is contemplated as capable of being included in the present compositions. In this regard, preferred non-limiting examples of retinoids useful in the present compositions include tazarotene, retinoic acid, tretinoin, isotretinoin, adapalene, bexarotene, alitretinoin, vitamin A, retinol, retinal, retinyl palmitate, retinyl acetate, ethyl 5-(2-(4,4-dimethylthiochroman-6-yl)ethynyl )thiophene-2-carboxylate, 6-(2-4,4-dimethylthiochroman-6-yl)-ethynyl)-3-pyridylmethanol, 2-(2-(4,4-dimethylthiochroman-6-yl)-ethynyl)-5-pyridinecarboxaldehyde, salts thereof, derivatives thereof, and mixtures thereof. Tazarotene, retinoic acid, tretinoin, and isotretinoin, as well as salts or derivatives thereof, particularly salts or derivatives of retinoic acid, tretinoin, or isotretinoin, are especially preferred in this regard. In a most preferred embodiment, the retinoid is tazarotene.

In preferred embodiments, one or more of the benzoyl peroxide, antibiotic or a pharmaceutically acceptable salt or ester thereof, and retinoid or a pharmaceutically acceptable salt thereof in the present compositions is encapsulated or entrapped in a solid or semi-solid ingredient for inclusion in the final compositions. This encapsulation or entrapment of the active ingredient(s) can help prevent reactions between the retinoid, antibiotic, and benzoyl peroxide components, thus promoting the storage-stability of each of these ingredients and of the composition as a whole.

Accordingly a preferred embodiment of the present subject matter additionally relates to a method for the treatment of acne in a patient in need thereof, comprising administering a combination of benzoyl peroxide, an antibiotic, and a retinoid to said patient, wherein said combination contains a low level of lincomycin phosphate sulfoxide, lincomycin sulfoxide, clindamycin phosphate sulfoxide, clindamycin sulfoxide, tazarotene sulfoxide, tazarotene sulfone, benzoic acid, and mixtures thereof.

In further preferred embodiments, this solid or semi-solid ingredient has a melting point at about a mammal's body temperature, such as a human's body temperature. Specific solid and semi-solid ingredients useful in this regard are well known to those of ordinary skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, http://www.accessdata.fda.gov/scripts/cder/iig/index.cfm, the contents of which are hereby incorporated by reference in their entirety.

Similarly, in a preferred embodiment one or more of the benzoyl peroxide, antibiotic or a pharmaceutically acceptable salt or ester thereof, and retinoid or a pharmaceutically acceptable salt thereof can be present in the final composition in a solution, suspension, or dispersion. In a particularly preferred embodiment in this regard, the benzoyl peroxide and the retinoid or a pharmaceutically acceptable salt thereof are in suspension and the antibiotic or a pharmaceutically acceptable salt or ester thereof is in solution.

Once all of the ingredients are combined, the preferred final compositions herein have a final viscosity of about 20,000 to about 1,000,000 centipoises. In a particularly preferred embodiment, the final compositions have a final viscosity of about 40,000 to about 500,000 centipoises. This final viscosity that is lower than the viscosity of the initial benzoyl peroxide-containing composition demonstrates that the present compositions are easier to mix together, contain less degradates, and have a greater degree of uniformity than those compositions previously known in the art.

In a preferred embodiment, the final compositions exhibit a final pH of about 3 to about 8. In a particularly preferred embodiment, the present compositions exhibit a final pH of about 3.5 to about 5.5. This narrowly tailored pH is in part responsible for the advanced storage stability of the present compositions in comparison to those previously known in the art. In this regard, the present preferred compositions can remain storage stable at a temperature of up to about 25° C. for at least 30 days, or more. In a particularly preferred embodiment, the present compositions can remain storage stable at a temperature of up to about 30° C. for at least 30 days. In another preferred embodiment, the present compositions can remain storage stable at a temperature of up to about 25° C. for at least 60 days.

In an alternative preferred embodiment, the present compositions can remain storage stable at a refrigerated temperature of not more than 15° C. for at least 60 days. In a particularly preferred embodiment in this regard, the present compositions can remain storage stable at a refrigerated temperature of about 2° C. to about 8° C. for at least 60 days. In an especially preferred embodiment, the present compositions can remain storage stable at a refrigerated temperature of about 2° C. to about 8° C. for at least 6 months. In another especially preferred embodiment, the present compositions can remain storage stable at a refrigerated temperature of about 2° C. to about 8° C. for at least 12 months.

In another alternative embodiment, the present compositions can remain storage stable under conditions selected from the group consisting of freezer conditions of less than about 0° C., about 2° C. to about 8° C., about 8° C. to about 15° C., about 23° C. to about 27° C., up to about 25° C., and about 15° C. to about 30° C.

The present preferred compositions do not require compounding at the time of dispensing and maintain stability for extended periods depending on the storage temperature, despite the relative incompatibility of the benzoyl peroxide, antibiotic, and retinoid. This represents a distinct advantage over the formulations presently known in the art.

The present compositions may be formulated for either once-per-day or twice-per-day administration. In a preferred embodiment, the once-per-day administration is in the evening or at night to increase compliance and to account for skin conditions most favorable to reducing inflammation.

### Pharmaceutically Acceptable Carrier

The initial benzoyl peroxide-containing composition, as well as the final composition, may take the form of a solution, gel, cream, lotion, suspension, emulsion, ointment, spray, foam, paste, or any combination thereof. Other cosmetic treatment compositions known to those skilled in the art, including liquids and balms, are additionally contemplated as falling within the scope of the present subject matter. Accordingly, the present compositions further include any pharmaceutically acceptable carrier suitable for providing the specific dosage form desired.

Emulsions, such as oil-in-water or water-in-oil systems, as well as a base (vehicle or carrier) for the topical formulation can be selected to provide effectiveness of the active ingredients and/or avoid allergic and irritating reactions (e.g., contact dermatitis) caused by ingredients of the base or by the active ingredients.

Accordingly, the present compositions may optionally further comprise an emulsifier. Non-limiting examples of emulsifiers useful in this regard include glycol esters, fatty acids, fatty alcohols, fatty acid glycol esters, fatty esters, fatty ethers, esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, derivatives thereof, and mixtures thereof.

In this regard, specific, non-limiting examples of emulsifiers useful in the present compositions include polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, tragacanth gum, poly(acrylamide-b-acrylic acid), 10-30 alkyl acrylate crosspolymers, derivatives thereof, and mixtures thereof.

Creams useful in the present compositions may also be semisolid emulsions of oil and water; are easily applied and vanish when rubbed into the skin.

Lotions useful in the present compositions include older definitions such as suspensions of powdered material (e.g., calamine) in a water or alcohol base, as well as modern lotions (e.g., some corticosteroids) such as water-based emulsions. Convenient to apply, lotions are also cool and help to dry acute inflammatory and exudative lesions.

Ointments which are useful are oleaginous and contain little if any water; feel greasy but are generally well tolerated; best used to lubricate, especially if applied over hydrated skin; they are preferred for lesions with thick crusts, lichenification, or heaped-up scales and may be less irritating than cream for some eroded or open lesions (e.g., stasis ulcers). Drugs in ointments are often more potent than in creams.

In a preferred embodiment, the present compositions may take the form of a gel. In this regard, the present compositions may include a gelling agent and/or a thickener. Suitable gelling agents and/or thickeners which may be useful in the present compositions include aqueous thickening agents, such as neutral, anionic, and cationic polymers, and mixtures thereof. Exemplary polymers which may be useful in the instant compositions include carboxy vinyl polymers, such as carboxypolymethylene. A preferred thickener is a carbomer, for example Carbopol® brand Carbopol polymer such as is available from Noveon Inc., Cleveland, Ohio. Other exemplary polymers useful in this regard include hydrophilic/hydrophobic graft copolymers, such as polymers formed as a mixture of polystyrene/microsponge/Carbopol®. One such polymer in this regard is a dimethylacrylamide/acrylic acid/polystyrene ethyl methacrylate copolymer, for example Pharmadur® brand copolymer such as is available from Polytherapeutics, Inc., Bridgewater, N.J.

Other, non-limiting example of suitable thickeners useful herein include cellulosic polymers, such as gum arabic, gum acacia, gum tragacanth, locust bean gum, guar gum, hydroxypropyl guar, xanthan gum, cellulose gum, sclerotium gum, carageenan gum, karaya gum, cellulose gum, rosin, methylcellulose, hydroxyethylcellulose, hydroxypropylcellulose, hydroxymethylcellulose, hydroxypropylmethylcellulose, methylhydroxyethylcellulose, cetyl hydroxyethylcellulose, carboxymethylcellulose, corn starch, hydroxypropyl starch phosphate, distarch phosphate, distarch dimethylene urea, aluminum starch octenyl succinate, maltodextrin, dextran, poly(acrylamide), PEG-150 distearate, PEG-150/decyl alcohol/SMDI copolymer, PEG-150/stearyl alcohol/SMDI copolymer, PEG-180/Laureth-50/TMMG copolymer, Polyether 1, acrylic acid/acrylamidomethyl propane sulfonic acid copolymer, acrylate/C10-30 alkyl acrylate crosspolymer, acrylate/beheneth-25 methacrylate copolymer, acrylate/steareth-20 methacrylate copolymer, acrylate/steareth-20 copolymer, acrylate/VA crosspolymer, acrylic acid/acrylonitrogen copolymer, ammonium acryloyldimethyltaurate/beheneth-25 methacrylate copolymer, ammonium acryloyldimethyltaurate/VP copolymer, caprylic/capric triglyceride (and) sodium acrylate copolymer, PVM/MA decadiene crosspolymer, alginic acid, propylene glycol alginate, dimethicone, silica dimethyl silylate, a dimethylacrylamide/acrylic acid/polystyrene ethyl methacrylate copolymer, derivatives thereof, and mixtures thereof. Other common thickeners and/or gelling agents, such as polyacrylic polymers, may be further useful herein. These thickeners and/or gelling agents can be present in the instant compositions regardless of what form the final composition takes.

Any other non-toxic, inert and effective carrier may be used to formulate the present preferred compositions. Well-known carriers used to formulate other therapeutic compounds for administration to humans particularly will be useful in the compositions of the present invention. Pharmaceutically acceptable carriers, excipients and diluents in this regard are well known to those of skill in the art, such as those described in The Merck Index, Thirteenth Edition, Budavari et al., Eds., Merck & Co., Inc., Rahway, N.J. (2001), which is incorporated by reference herein in its entirety. Examples of such useful pharmaceutically acceptable excipients, carriers and diluents include distilled water, physiological saline, Ringer's solution, dextrose solution, Hank's solution and DMSO, which are among those preferred for use in the present invention.

These additional components, as well as effective formulations and administration procedures are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

Examples of preferred excipients that can be used according to the present preferred compositions include but are not limited to a carbomer, a polyacrylic polymer, glycerin, sodium hydroxide, sodium thiosulfate, propyl gallate, an alkyl paraben, purified water, and mixtures thereof.

Other ingredients which may optionally be provided in the instant topical compositions include humectants, such as propylene glycol; solvents, such as alcohol (de minimis); sun filters, such as titanium dioxide, zinc oxide, and calcium carbonate; and anti-microbial preservatives, such as methylparaben and propylparaben. The topical compositions may also include an organic or inorganic base, such as sodium hydroxide, which is used to adjust the pH of the initial components and the final product.

In this regard, the preferred compositions discussed herein can additionally comprise remaining amounts of one or more dermatologically acceptable excipients. Preferred, non-limiting examples of dermatologically acceptable excipients useful in these compositions are those selected from the group consisting of surfactants, preservatives, emollients, humectants, fluid alkyl alcohols, thickening agents, emulsifiers, suspending agents, pH modifiers/buffering agents, chelating agents, sun filters, derivatives thereof, and mixtures thereof. Although the present compositions may optionally further contain an antioxidant as one of the dermatologically acceptable excipients, the use of an antioxidant in this regard is not particularly preferred.

Accordingly, any surfactant, preservative, emollient, humectant, fluid alkyl alcohol, thickening agent, emulsifier, suspending agent, pH modifier, chelating agent, antioxidant, sun filter, or other dermatologically acceptable excipient commonly known to those of ordinary skill in the art as useful in topical compositions is contemplated as useful in the compositions described herein. Further, any non-toxic, inert, and effective topical carrier may be used to formulate the compositions described herein. Well-known carriers used to formulate other topical therapeutic compositions for administration to humans will be useful in these compositions. Examples of these components that are well known to those of skill in the art are described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, http://www.accessdata.fda.gov/scripts/cder/iig/index.cfm, the contents of which are hereby incorporated by reference in their entirety.

In severe cases, occlusive therapy may be useful where acne is present concurrently with other indications or conditions such as psoriasis, atopic dermatitis, lupus erythematosus, and chronic hand dermatitis. Covering the treated area with a nonporous occlusive dressing can increase the absorption and effectiveness of the present compositions. Usually, a polyethylene film (plastic household wrap) is applied overnight over cream or ointment, which tends to be less irritating than lotion for occlusive therapy. Plastic tape may be impregnated with drug and is especially convenient for treating isolated or recalcitrant lesions; children and (less often) adults may experience pituitary and adrenal suppression after prolonged occlusive therapy over large areas.

In an alternative embodiment, the present subject matter further relates to a topical composition for treating a skin disorder or condition, which comprises a storage-stable mixture of 1) a retinoid or a pharmaceutically acceptable salt thereof, 2) either a benzoyl peroxide-containing composition or an antibiotic or a pharmaceutically acceptable salt or ester thereof, and 3) a pharmaceutically acceptable carrier. In a preferred embodiment, the composition is formulated so that it has a final pH of about 3 to about 8.

### Additional Active Ingredients

In addition to the benzoyl peroxide, antibiotic, and retinoid, the present compositions may further contain other active ingredients readily known to those of skill in the art as useful in the topical treatment of skin disorders or conditions. Exemplary additional active ingredients include, but are not limited to, other macrolide antibiotics, bactericidal drugs, bacteriostatic drugs, cleansing agents, absorbents, anti-infective agents, anti-inflammatory agents, astringents (drying agents that precipitate protein and shrink and contract the skin), emollients (skin softeners), moisturizers, keratolytics (agents that soften, loosen, and facilitate exfoliation of the squamous cells of the epidermis), and mixtures thereof.

Exemplary additional macrolide antibiotics contemplated as within the scope of the present subject matter include, but are not limited to, Azithromycin, Clarithromycin, Erythromycin, Lincomycin, and mixtures thereof. The macrolides are similar in structure and activity. All the macrolides are easily absorbed and all are primarily bacteriostatic and bind to the 50S subunit of the ribosome, thus inhibiting bacterial protein synthesis. These drugs are typically active against aerobic and anaerobic gram-positive cocci, with the exception of enterococci, and against gram-negative anaerobes and useful in the present compositions.

Exemplary bactericidal drugs (i.e., they kill bacteria) contemplated as within the scope of the present subject matter include, but are not limited to, Penicillins, cephalosporins, vancomycin, aminoglycosides, quinolones, and polymyxins.

Exemplary bacteriostatic drugs (i.e., they slow bacterial growth) contemplated as within the scope of the present subject matter include, but are not limited to, erythromycin, tetracyclines, chloramphenicol, lincomycin, clarithromycin, azithromycin, and sulfonamides. However, it is well know that some bactericidal drugs may be bacteriostatic against certain microorganisms and vice versa. These drugs are well known in the art and may be found, for example, in The Merck Manual of Diagnosis and Therapy, 13^{th} edition, Section 13, Chapter 153 Anti-bacterial Drugs, 2001, incorporated herein by reference in its entirety.

In another preferred embodiment, the present compositions may be used in combination with an additional pharmaceutical dosage form to enhance their effectiveness in treating a skin disease, disorder, or condition. In this regard, the present compositions may be administered as part of a regimen additionally including any other pharmaceutical and/or pharmaceutical dosage form known in the art as effective for the treatment of a skin disorder. Accordingly, the additional active ingredient or additional pharmaceutical dosage form can be applied to a patient either directly or indirectly, and concomitantly or sequentially, with the preferred compositions described herein.

In one embodiment in this regard, the present composition and the additional pharmaceutical dosage form can be administered to a patient at the same time. In an alternative embodiment, one of the present compositions and the additional pharmaceutical dosage form can be administered in the morning and the other can be administered in the evening.

In another preferred embodiment, the additional pharmaceutical dosage form can be an oral pharmaceutical dosage form. In this regard, the present topical dosage form can be applied to the target area of the patient, prior to, concomitantly with, or after ingestion of the oral medication.

Furthermore, the formulation may be used with other adjunct therapies and treatments, such as pre-washing with common soaps, and mild detergents. However, selection is important when treating skin disorders such as acne since antibacterial soaps and abrasive soaps may increase irritation and make it difficult to use follicular drugs. Such follicular drugs may include topical antibiotics and antiseptics, as well as intralesional corticosteroids.

In superficial pustular acne, the topical benzoyl peroxide/antibiotic/retinoid compositions may be used in combination with one of the follicular drugs.

Another combination therapy involves Azelaic acid cream 20%, which has antiproliferative and antibacterial effects, and is known to be effective in comedonal or inflammatory acne.

Other topical drugs including OTC drugs, various sulfur-resorcinol combinations, and oral antibiotics may also be helpful in combination with the present compositions when treating superficial pustular acne.

### Methods of Use

The present subject matter also relates to a method for treating a skin disorder or condition in a patient by topically administering to a patient in need thereof one of the above-described topical compositions in an amount effective to treat the skin disorder.

Skin disorders or conditions treatable according to the present methods include but are not limited to microbial infections and inflammation of tissue. The microbial infections can be caused by gram-positive bacteria, gram-negative bacteria, and combinations thereof. Exemplary specific bacteria treatable by the present compositions include but are not limited to P. acnes, Strep. pyogenes, E. coli, Pseudomonas aeruginosa, Staph. aureus, and combinations thereof.

Exemplary, non-limiting specific skin disorders, diseases, or conditions treatable by the present compositions include but are not limited to acne, impetigo, rosacea, psoriasis, dermatitis, secondary skin infections, responsive dermatoses, and combinations thereof. Other specific skin disorders treatable by the present compositions include seborrhea, skin lesions, atopic dermatitis, and bacterial skin infections. In a preferred embodiment, the skin disorder or condition improves following treatment with the present compositions.

In another preferred embodiment, the present subject matter further relates to a method for the treatment of acne in a patient in need thereof, comprising administering a combination of benzoyl peroxide, an antibiotic, and a retinoid which has been refrigerated to said patient. This combination has a specific degradation profile, in accordance with the data submitted below.

In a preferred embodiment, the present compositions are intended for treatment of both pediatric and adult patients. In this regard, the pediatric patient will typically be up to 18 years old. In another preferred embodiment, the adult patient to be treated is between the ages of 19 and 85. In a particularly preferred embodiment, the adult patient to be treated is between the ages of 19 and 45. In yet another preferred embodiment, the patient to be treated is between the ages of 19 and 25.

### Process for Preparing

The present subject matter further relates to a process for preparing a topical composition comprising a storage-stable mixture of a benzoyl peroxide suspension, an antibiotic or a pharmaceutically acceptable salt thereof, a retinoid or a pharmaceutically salt thereof, and a pharmaceutically acceptable carrier.

The present preferred processes can be carried out in various steps. One preferred step requires separately preparing a benzoyl peroxide intermediate composition, a retinoid intermediate composition, and an antibiotic solution, each of which is prepared at a temperature of about 15 to about 30° C.

In a preferred process step, the pH of the benzoyl peroxide intermediate composition may be adjusted before it is mixed with the retinoid intermediate composition and antibiotic solution under conditions sufficient to yield a topical composition having a final pH of between about 3 to about 8. Preferably, the composition thus formed comprises sufficient inactive ingredients to provide storage stability and effectiveness for a treatment period.

In an alternative embodiment, the retinoid present in the composition can be encapsulated or entrapped. This is preferably done in a solid or semi-solid, which can have a melting point at about a mammal's body temperature. In further alternative embodiments, either of the benzoyl peroxide present in the benzoyl peroxide intermediate composition or the antibiotic present in the antibiotic solution, or any combination of these three or any mixture thereof, can be encapsulated or entrapped in a solid or semi-solid material prior to mixing. This encapsulation or entrapment step can promote the storage-stability of the topical composition.

In a further alternative embodiment, the benzoyl peroxide intermediate composition and the retinoid intermediate composition can be separately milled prior to their mixing with the antibiotic solution.

In a preferred embodiment, the final composition made according to the present process will have a viscosity lower than the viscosity of the benzoyl peroxide intermediate composition.

The present processes can preferably result in compositions having benzoyl peroxide impurities or degradates of not more than about 0.15% by weight, antibiotic impurities or degradates of not more than about 0.01% by weight, and retinoid impurities or degradates of not more than about 0.001% by weight at the time the composition is made or produced.

The combinations produced according to these processes can maintain stability for a minimum of one month at room temperature (e.g. 22° C.) and relative, or ambient, humidity.

### Routes of Administration/Dosage

To be effective, the route of administration for the compositions used in the present methods and pharmaceutical compositions must readily affect the target areas. In particular, acne is known to affect the face, neck, back, ears, and scalp.

Dosage levels for the antibiotic, the benzoyl peroxide, and the retinoid are well known in the art and are selected to maximize the treatment of the above conditions. The specific dose level for any particular patient will vary depending upon a variety of factors, including the activity of the specific compound employed; the age, body weight, general health, sex and diet of the patient; the time of administration; the rate of excretion; drug combination; the severity of the particular disease being treated; and the form of administration. Typically, in vitro dosage-effect results can provide useful guidance on the proper doses for patient administration. Studies in animal models are also helpful. The considerations for determining the proper dose levels are well known in the art and are incorporated herein for the present subject matter.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time are well known in the art.

Lessening exposure by once-daily administration affects multiple pharmacokinetic parameters and provides the initial mechanism for avoiding skin irritation and inflammation and the other toxicity issues discussed herein. Additional formulations may be prepared which factor in the benefit/risk ratio for an antibiotic, benzoyl peroxide, and retinoid composition. The level of toxicity of these compounds is known and reference is made to the package inserts for Cleocin T® and BenzaClin® and the level of adverse events reported from their clinical trials, which package inserts are hereby specifically incorporated by reference in their entirety. In particular, BenzaClin® reported having the following events: dry skin (12%), pruritis (2%), peeling (2%), erythema (1%) and sunburn (1%) as compared to vehicle which reported dry skin (6%), pruritis (<1%), peeling (−), erythema (<1%) and sunburn (−), or roughly twice the number of side effects as vehicle.

Since benzoyl peroxide is a keratolytic, i.e. causes softening and swelling of the cells at the surface of the skin so that the outer layer of the skin peels off or can easily be removed, reducing exposure to it reduces irritation. Upon application, the benzoyl peroxide converts to benzoic acid and has anti-bacterial and anti-fungal properties. Additionally, the low pH of the present formulations may have an additive keratolytic effect on the skin as well as on the anti-bacterial properties. Benzoyl peroxide may also act as a preservative within the formulation. The antibiotic, such as clindamycin, may degrade at pH higher than about 6.5, thus requiring the pH to be maintained below this level, as described herein. The present formulations take these and other factors into account and are manufactured to reduce sensitivity, irritation, and/or inflammation.

Single dosage kits and packages containing once per day amounts of the present compositions may be prepared. Single dose, unit dose, and once-daily disposable containers of the mixtures and compositions herein are contemplated as within the scope of the present subject matter.

The present compositions may be formulated for storage in a substantially non-reactive package to enhance stability of the product. This new method of storage provides enhanced product stability in comparison with the previous paper-based packages. Non-limiting examples of preferred non-reactive packages in this regard include a glass package, a molded or flexible plastic package, a single-dose vial, an aluminum package, a tin package, a composite cardboard package, a laminated package, a laminated pouch, a pump, and a combination thereof. Composite cardboard packages useful in this regard include wax coated cardboard packages.

In preferred embodiments, the composition can be stored in the non-reactive package under a blanket of an inert gas. Preferred, non-limiting examples of inert gases useful in this regard include nitrogen gas, argon gas, and a mixture thereof.

Additionally, the use of one of these packaging systems permits the present compositions to be stored such that a combination of any two of the initial benzoyl peroxide-containing composition, the antibiotic or a pharmaceutically acceptable salt or ester thereof, and the retinoid or a pharmaceutically acceptable salt thereof are stable at room temperature. In an alternative preferred embodiment, at least two of the initial benzoyl peroxide-containing composition, the antibiotic or a pharmaceutically acceptable salt or ester thereof, and the retinoid or a pharmaceutically acceptable salt thereof require refrigeration.

The amount of composition per single packet may range from about 0.1 mL to about 20.0 mL, preferably between about 0.5 and about 5.0 mL, more preferably between about 1 and about 3 mL.

In another alternative embodiment, the present compositions can be administered using an applicator. Non-limiting examples of useful applicators in this regard include a pledget, a swab, a pad, and combinations thereof. These useful applicators can be made from any material known to those of ordinary skill in the art as useful in this regard, including but not limited to polyurethane foam, rayon, polyethylene, polypropylene, cotton, polyesters, and combinations thereof. Additionally, the present subject matter further contemplates that any of these topical compositions are provided in a package of less than 5 g topical composition as a unit of use.

The ability to formulate compositions capable of long term storage, without pre-mixing or compounding requirements prior to application, are also contemplated herein. Specifically, the present compositions remain unexpectedly stable in storage for periods including between about 2 weeks and about 18 months, preferably between about 3 weeks and about 15 months, more preferably between about 30 days and about 12 months.

Once-daily disposable packaging may also improve patient compliance, especially for teenagers.

The stability and effectiveness of the topical preparations may last for at least 1 to 18 months at ambient or room temperature. Stability is maintained under refrigeration for an extended period of time because degradation is slowed through the storage temperature. This improved stability provides pharmacists and other dispensers of medication with a product which no longer requires compounding at the time of dispensing. Because compounding is no longer required, homogeneity is controlled at the point of manufacture, which improves dosing and ultimately compliance.

Advantageously, the final product requires no compounding by the pharmacist. In addition, compliance with exact amounts is possible with a lessened chance of impurities entering the product and contaminating it.

By maintaining the compositions at the present specific pH, the tendency of benzoyl peroxide to oxidize and degrade the antibiotic and retinoid is largely overcome and the product remains stable during storage at room temperature for extended periods.

The following examples are illustrative of preferred embodiments herein and are not to be construed as limiting the present subject matter thereto. All polymer molecular weights are mean average molecular weights. All percentages are based on the percent by weight of the final delivery system or formulation prepared unless otherwise indicated and all totals equal 100% by weight.

### EXAMPLE 1

A benzoyl peroxide-containing composition, a tazarotene-containing composition, and a clindamycin solution are mixed together to prepare a final composition having the following components.

| Ingredient | Percent by Weight |
|---|---|
| Benzoyl Peroxide 78% | 6.73 |
| Clindamycin phosphate | 1.28 |
| Tazarotene | 0.10 |
| Purified Water | 85.875 |
| Glycerin | 5.00 |
| Carbopol 980 | 0.85 |
| Sodium Hydroxide | 0.075 |
| Titanium Dioxide | 0.05 |
| Methylparaben | 0.04 |
| Total: | 100.0% |

### EXAMPLE 2

A benzoyl peroxide-containing composition, a tazarotene-containing composition, and a clindamycin solution are mixed together to prepare a final composition having the following components.

| Ingredient | Percent by Weight |
|---|---|
| Benzoyl Peroxide Hydrous | 1.33 |
| Clindamycin phosphate | 1.28 |
| Tazarotene | 0.10 |
| Purified Water | 91.27 |
| Glycerin | 5.00 |
| Carbopol 980 | 0.85 |
| Sodium Hydroxide | 0.08 |
| Titanium Dioxide | 0.05 |
| Methylparaben | 0.04 |
| Total: | 100.0% |

### EXAMPLE 3

A benzoyl peroxide-containing composition, a tretinoin-containing composition, and a clindamycin solution are combined to prepare a final composition having the following components.

| Ingredient | Percent by Weight |
|---|---|
| Purified Water | 89.82 |
| Benzoyl Peroxide Hydrous | 2.84 |
| Clindamycin phosphate | 1.28 |
| Tretinoin | 0.102 |
| Glycerin | 4.99 |
| Carbopol 980 | 0.84 |
| Sodium Hydroxide | 0.088 |
| Methylparaben | 0.04 |
| Total: | 100.0% |

### EXAMPLE 4

A benzoyl peroxide-containing composition, a tretinoin-containing composition, and a clindamycin solution are combined to prepare a final composition having the following components.

| Ingredient | Percent by Weight |
|---|---|
| Purified Water | 85.05 |
| Benzoyl Peroxide 75% | 2.67 |
| Clindamycin phosphate | 1.28 |
| Tretinoin | 0.10 |
| Glycerin | 9.89 |
| Carbopol 980 | 0.84 |
| Sodium Hydroxide | 0.08 |
| Methylparaben | 0.04 |
| Titanium Dioxide | 0.05 |
| Total: | 100.0% |

### EXAMPLE 5

A benzoyl peroxide-containing composition, an adapalene-containing composition, and a clindamycin solution are combined to prepare a final composition having the following components.

| Ingredient | Percent by Weight |
|---|---|
| Purified Water | 85.05 |
| Benzoyl Peroxide 75% | 2.67 |
| Clindamycin phosphate | 1.28 |
| Adapalene | 0.10 |
| Glycerin | 9.89 |
| Carbopol 980 | 0.84 |
| Sodium Hydroxide | 0.08 |
| Methylparaben | 0.04 |
| Titanium Dioxide | 0.05 |
| Total: | 100.0% |

### EXAMPLE 6

Tables 1, 2, and 3 show the stability of the active ingredients. An analysis was performed on a composition containing 5.54% of benzoyl peroxide, 0.101% of tazarotene, and 1.06% of clindamycin. Measurements were taken at the end of 2 weeks and 90 days. The composition was stored at 4 different temperatures, i.e., 6° C., 25° C., 30° C., and 40° C. The levels of benzoyl peroxide, tazarotene, and clindamycin were measured at each temperature. The results are as follows:

TABLE 1
Benzoyl Peroxide (BPO) (as % w/w): Initial 5.54%

| | 6° C. | 25° C. | 30° C. | 40° C. |
|---|---|---|---|---|
| 2 weeks | n/a | 5.43 | 5.35 | 5.34 |
| 90 days | 5.48 | 5.34 | 5.33 | 5.19 |

TABLE 2
Tazarotene (as % w/w): Initial 0.101%

| | 6° C. | 25° C. | 30° C. | 40° C. |
|---|---|---|---|---|
| 2 weeks | n/a | 0.088 | 0.085 | 0.081 |
| 90 days | 0.094 | 0.090 | 0.085 | 0.078 |

TABLE 3
Clindamycin (as % w/w): Initial 1.06%

| | 6° C. | 25° C. | 30° C. | 40° C. |
|---|---|---|---|---|
| 2 weeks | n/a | 1.06 | 1.06 | 0.90 |
| 90 days | 1.04 | 1.01 | 0.96 | 0.72 |

### EXAMPLE 7

Tables 4, 5, and 6 show the stability of the active ingredients in a composition containing 5.22% of benzoyl peroxide, 0.103% of tazarotene, and 1.06% of clindamycin.

A 2-week and 6 month analysis of the composition was undertaken following the procedure of Example 4.

TABLE 4
Clindamycin (as % w/w): Initial 1.06%

| | 6° C. | 25° C. | 30° C. | 40° C. |
|---|---|---|---|---|
| 2 weeks | 1.06 | 1.06 | 1.05 | 1.00 |
| 6 months | 1.00 | 0.90 | 0.91 | 0.42 |

TABLE 5
Benzoyl Peroxide (BPO) (as % w/w): Initial 5.22%

| | 6° C. | 25° C. | 30° C. | 40° C. |
|---|---|---|---|---|
| 2 weeks | 5.22 | 5.24 | 5.23 | 5.12 |
| 6 months | 5.16 | 4.80 | 4.91 | 4.59 |

TABLE 6
Tazarotene (as % w/w): Initial 0.103%

| | 6° C. | 25° C. | 30° C. | 40° C. |
|---|---|---|---|---|
| 2 weeks | 0.104 | 0.098 | 0.088 | 0.080 |
| 6 months | 0.106 | 0.090 | 0.082 | 0.046 |

### EXAMPLE 8

Tables 16, 17, and 18 show the stability of the active ingredients in a composition containing 2.15% of benzoyl peroxide, 0.103% of tazarotene, and 1.07% of clindamycin.

A 6 month analysis of the composition was undertaken following the procedure of Example 4.

TABLE 16
Clindamycin (as % w/w): Initial 1.07%

| | 6° C. | 25° C. | 30° C. | 40° C. |
|---|---|---|---|---|
| 6 months | 1.01 | 0.91 | 0.86 | 0.43 |

TABLE 17
Benzoyl Peroxide (BPO) (as % w/w): Initial 2.15%

| | 6° C. | 25° C. | 30° C. | 40° C. |
|---|---|---|---|---|
| 6 months | 2.13 | 2.00 | 2.05 | 1.69 |

TABLE 18
Tazarotene (as % w/w): Initial 0.103%

| | 6° C. | 25° C. | 30° C. | 40° C. |
|---|---|---|---|---|
| 6 months | 0.099 | 0.097 | 0.079 | 0.057 |

### EXAMPLE 9

Tables 19, 20, and 21 show the stability of the active ingredients in a composition containing 2.02% of benzoyl peroxide, 0.106% of tazarotene, and 1.06% of clindamycin.

A 90 day and 6 month analysis of the composition was undertaken following the procedure of Example 4.

TABLE 19
Clindamycin (as % w/w): Initial 1.06%

| | 6° C. | 25° C. | 30° C. | 40° C. |
|---|---|---|---|---|
| 90 days | 1.04 | 0.99 | 0.97 | 0.67 |
| 6 months | 1.04 | 0.941 | 0.894 | 0.325 |

TABLE 20
Benzoyl Peroxide (BPO) (as % w/w): Initial 2.02%

| | 6° C. | 25° C. | 30° C. | 40° C. |
|---|---|---|---|---|
| 90 days | 2.02 | 1.97 | 1.97 | 1.73 |
| 6 months | 2.10 | 2.00 | 1.94 | 1.45 |

TABLE 21
Tazarotene (as % w/w): Initial 0.106%

| | 6° C. | 25° C. | 30° C. | 40° C. |
|---|---|---|---|---|
| 90 days | 0.103 | 0.092 | 0.090 | 0.071 |
| 6 months | 0.104 | 0.089 | 0.089 | 0.003 |

### EXAMPLE 10

Tables 22, 23, and 24 show the stability of the active ingredients in a composition containing 1.54% of benzoyl peroxide, 0.105% of tazarotene, and 1.04% of clindamycin.

A 90 day and 6 month analysis of the composition was undertaken following the procedure of Example 4.

TABLE 22
Clindamycin (as % w/w): Initial 1.04%

| | 6° C. | 25° C. | 30° C. | 40° C. |
|---|---|---|---|---|
| 90 days | 1.04 | 0.99 | 0.98 | 0.72 |
| 6 months | 1.03 | 0.92 | 0.90 | 0.41 |

TABLE 23
Benzoyl Peroxide (BPO) (as % w/w): Initial 1.54%

| | 6° C. | 25° C. | 30° C. | 40° C. |
|---|---|---|---|---|
| 90 days | 1.50 | 1.44 | 1.42 | 1.28 |
| 6 months | 1.50 | 1.40 | 1.40 | 1.03 |

TABLE 24
Tazarotene (as % w/w): Initial 0.105%

| | 6° C. | 25° C. | 30° C. | 40° C. |
|---|---|---|---|---|
| 90 days | 0.106 | 0.088 | 0.088 | 0.076 |
| 6 months | 0.101 | 0.091 | 0.085 | 0.014 |

### EXAMPLE 11

Tables 7, 8, and 9 show the stability of the active ingredients in a composition containing 1.01% of benzoyl peroxide, 0.104% of tazarotene, and 1.1% of clindamycin.

A 2 week, 30 day, 90 day, and 6 month analysis of the composition was undertaken following the procedure of Example 4.

TABLE 7
Clindamycin (as % w/w): Initial 1.10%

| | 6° C. | 25° C. | 40° C. |
|---|---|---|---|
| 2 weeks | 1.07 | 1.07 | 1.01 |
| 30 days | 1.08 | 1.07 | 0.95 |
| 90 days | 1.07 | 1.04 | 0.71 |
| 6 months | 1.07 | 0.99 | 0.53 |

TABLE 8
Benzoyl Peroxide (BPO) (as % w/w): Initial 1.01%

| | 6° C. | 25° C. | 40° C. |
|---|---|---|---|
| 2 weeks | 0.99 | 0.95 | 0.94 |
| 30 days | 0.98 | 0.95 | 0.90 |
| 90 days | 0.99 | 0.93 | 0.69 |
| 6 months | 0.99 | 0.91 | 0.35 |

TABLE 9
Tazarotene (as % w/w): Initial 0.104%

| | 6° C. | 25° C. | 40° C. |
|---|---|---|---|
| 2 weeks | 0.106 | 0.098 | 0.089 |
| 30 days | 0.105 | 0.094 | 0.084 |
| 90 days | 0.106 | 0.093 | 0.086 |
| 6 months | 0.107 | 0.093 | 0.014 |

### EXAMPLE 12

Tables 10, 11, and 12 show the stability of the active ingredients in a composition containing 1.02% of benzoyl peroxide, 0.099% of tazarotene, and 1.05% of clindamycin.

A 2-week, 90 day, and 6 month analysis of the composition was undertaken following the procedure of Example 4.

TABLE 10
Clindamycin (as % w/w): Initial 1.05%

| | 6° C. | 25° C. | 30° C. | 40° C. |
|---|---|---|---|---|
| 2 weeks | 1.05 | 1.05 | 1.04 | 0.98 |
| 90 days | 1.05 | 1.00 | 0.95 | 0.71 |
| 6 months | 1.05 | 0.97 | 0.89 | 0.44 |

TABLE 11
Benzoyl Peroxide (BPO) (as % w/w): Initial 1.02%

| | 6° C. | 25° C. | 30° C. | 40° C. |
|---|---|---|---|---|
| 2 weeks | 0.99 | 0.97 | 0.97 | 0.94 |
| 90 days | 1.00 | 0.94 | 0.93 | 0.73 |
| 6 months | 1.01 | 0.95 | 0.92 | 0.40 |

TABLE 12
Tazarotene (as % w/w): Initial 0.099%

| | 6° C. | 25° C. | 30° C. | 40° C. |
|---|---|---|---|---|
| 2 weeks | 0.099 | 0.090 | 0.090 | 0.085 |
| 90 days | 0.102 | 0.090 | 0.089 | 0.081 |
| 6 months | 0.096 | 0.091 | 0.088 | None Detected |

### EXAMPLE 13

Tables 13, 14, and 15 show the stability of the active ingredients in a composition containing 1.04% of benzoyl peroxide, 0.098% of tazarotene, and 1.06% of clindamycin.

A 2-week, 90 day, and 6 month analysis of the composition was undertaken following the procedure of Example 4.

TABLE 13
Clindamycin (as % w/w): Initial 1.06%

| | 6° C. | 25° C. | 30° C. | 40° C. |
|---|---|---|---|---|
| 2 weeks | 1.06 | 1.05 | 1.05 | 0.90 |
| 90 days | 1.06 | 1.01 | 0.95 | 0.71 |
| 6 months | 1.06 | 0.97 | 0.88 | 0.43 |

TABLE 14
Benzoyl Peroxide (BPO) (as % w/w): Initial 1.04%

| | 6° C. | 25° C. | 30° C. | 40° C. |
|---|---|---|---|---|
| 2 weeks | 0.99 | 0.98 | 0.98 | 0.96 |
| 90 days | 1.02 | 0.99 | 0.95 | 0.73 |
| 6 months | 1.02 | 0.96 | 0.91 | 0.40 |

TABLE 15
Tazarotene (as % w/w): Initial 0.098%

| | | | | |
|---|---|---|---|---|
| 2 weeks | 0.097 | 0.089 | 0.086 | 0.081 |
| 90 days | 0.100 | 0.090 | 0.087 | 0.079 |
| 6 months | 0.098 | 0.085 | 0.085 | None Detected |

### EXAMPLE 14

Tables 25 and 26 show the stability of the active ingredients in a composition containing 0.209% of tazarotene and 2.13% of clindamycin.

A 4-week, 90 day, and 6 month analysis of the composition was undertaken following the procedure of Example 4.

TABLE 25
Clindamycin (as % w/w): Initial 2.13%

| | 25° C. | 30° C. | 40° C. |
|---|---|---|---|
| 4 weeks | 2.10 | 2.10 | 2.04 |
| 90 days | 2.12 | 2.11 | 1.94 |
| 6 months | 2.07 | 1.96 | 1.74 |

TABLE 26
Tazarotene (as % w/w): Initial 0.209%

| | 25° C. | 30° C. | 40° C. |
|---|---|---|---|
| 4 weeks | 0.208 | 0.208 | 0.205 |
| 90 days | 0.204 | 0.208 | 0.208 |
| 6 months | 0.206 | 0.206 | 0.205 |

### EXAMPLE 15

Tables 27 and 28 show the stability of the active ingredients in a composition containing 0.109% of tazarotene and 1.07% of clindamycin.

A 60 day and 90 day analysis of the composition was undertaken following the procedure of Example 4.

TABLE 27
Clindamycin (as % w/w): Initial 1.07%

| | 6° C. | 25° C. | 30° C. | 40° C. |
|---|---|---|---|---|
| 60 days | 1.07 | 1.06 | 1.05 | 1.00 |
| 90 days | 1.05 | 1.04 | 1.04 | 0.96 |

TABLE 28
Tazarotene (as % w/w): Initial 0.109%

| | 6° C. | 25° C. | 30° C. | 40° C. |
|---|---|---|---|---|
| 60 days | 0.11 | 0.109 | 0.105 | 0.107 |
| 90 days | 0.107 | 0.111 | 0.108 | 0.116 |

### EXAMPLE 16

Tables 29 and 30 show the stability of the active ingredients in a composition containing 0.105% of tazarotene and 1.07% of clindamycin.

A 6 month analysis of the composition was undertaken following the procedure of Example 4.

TABLE 29
Clindamycin (as % w/w): Initial 1.07%

| | 6° C. | 25° C. | 30° C. | 40° C. |
|---|---|---|---|---|
| 6 months | 1.060 | 1.020 | 0.950 | 0.870 |

TABLE 30
Tazarotene (as % w/w): Initial 0.105%

| | 6° C. | 25° C. | 30° C. | 40° C. |
|---|---|---|---|---|
| 6 months | 0.101 | 0.102 | 0.104 | 0.103 |

### EXAMPLE 17

Tables 31 and 32 show the stability of the active ingredients in a composition containing 2.02% of benzoyl peroxide and 0.099% of tazarotene.

A 90 day and 6 month analysis of the composition was undertaken following the procedure of Example 4.

TABLE 31
Benzoyl peroxide (as % w/w): Initial 2.02%

| | 6° C. | 25° C. | 30° C. | 40° C. |
|---|---|---|---|---|
| 90 days | 2.09 | 2.06 | 2.07 | 2.01 |
| 6 months | 2.07 | 2.03 | 2.05 | 1.94 |

TABLE 32
Tazarotene (as % w/w): Initial 0.099%

| | 6° C. | 25° C. | 30° C. | 40° C. |
|---|---|---|---|---|
| 90 days | 0.106 | 0.088 | 0.085 | 0.044 |
| 6 months | 0.108 | 0.081 | 0.070 | 0.009 |

### EXAMPLE 18

Tables 33 and 34 show the stability of the active ingredients in a composition containing 5.18% of benzoyl peroxide and 0.099% of tazarotene.

A 90 day and 6 month analysis of the composition was undertaken following the procedure of Example 4.

TABLE 33
Benzoyl peroxide (as % w/w): Initial 5.18%

| | 6° C. | 25° C. | 30° C. | 40° C. |
|---|---|---|---|---|
| 90 days | 5.25 | 5.23 | 5.30 | 5.16 |
| 6 months | 5.2 | 5.14 | 5.02 | 4.81 |

TABLE 34
Tazarotene (as % w/w): Initial 0.099%

| | 6° C. | 25° C. | 30° C. | 40° C. |
|---|---|---|---|---|
| 90 days | 0.103 | 0.093 | 0.087 | 0.051 |
| 6 months | 0.106 | 0.088 | 0.083 | 0.004 |

### EXAMPLE 19

A patient is suffering from acne. A preferred composition herein is topically administered to the patient. It would be expected that the patient would improve his/her condition or recover.

The present subject matter being thus described, it will be obvious that the same may be varied in many ways. Such variations are not to be regarded as a departure from the spirit and scope of the invention and all such modifications are intended to be included within the scope of the following claims.

We claim:

1. A topical composition for treating a skin disorder or condition, which comprises:
a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,
wherein the composition has a final pH of about 3 to about 8, and wherein the composition has a viscosity lower than the viscosity of the benzoyl peroxide-containing composition before obtaining said storage-stable mixture.

2. The composition of claim 1, wherein said composition is formulated for once-per-day or twice-per-day administration.

3. The composition of claim 1, wherein said composition is formulated for once-per-day administration at night.

4. The composition of claim 1, wherein said composition has a final pH of about 3.5 to about 5.5.

5. The composition of claim 1, wherein said composition is selected from the group consisting of a solution, gel, cream, lotion, suspension, emulsion, ointment, spray, foam, paste, and mixtures thereof.

6. The composition of claim 1, wherein said composition is storage stable at a temperature of up to about 25° C. for at least 30 days.

7. The composition of claim 1, wherein said composition has a final viscosity of about 20,000 to about 1,000,000 centipoises.

8. The composition of claim 7, wherein said composition has a final viscosity of about 40,000 to about 500,000 centipoises.

9. The composition of claim 1, wherein said benzoyl peroxide-containing composition has a viscosity of about 25,000 to about 1,250,000 centipoises.

10. The composition of claim 1, wherein said mixture comprises about 0.1% to about 10% by weight of said benzoyl peroxide.

11. The composition of claim 1, wherein said mixture comprises about 0.5% to about 3% by weight of said antibiotic.

12. The composition of claim 1, wherein said mixture comprises about 0.01% to about 1.5% by weight of said retinoid.

13. The composition of claim 1, wherein said retinoid is selected from the group consisting of tazarotene, retinoic acid, tretinoin, isotretinoin, adapalene, bexarotene, alitretinoin, vitamin A, retinol, retinal, retinyl palmitate, retinyl acetate, ethyl 5-(2-(4,4-dimethylthiochroman-6-yl)ethynyl)thiophene-2-carboxylate, 6-(2-4,4-dimethylthiochroman-6-yl)-ethynyl)-3-pyridylmethanol, 2-(2-(4,4-dimethylthiochroman-6-yl)-ethynyl)-5-pyridinecarboxaldehyde, salts thereof, derivatives thereof, and mixtures thereof.

14. The composition of claim 1, wherein said antibiotic is selected from the group consisting of clindamycin, tetracycline, erythromycin, lincomycin, azithromycin, carbomycin, chlortetracycline, clarithromycin, demeclocycline, doxycycline, gentamicin, josamycin, kanamycin, leucomycins, meomycin, methacycline, midecamycins, miokamycin, oleandomycin, oxytetracycline, primycin, ribostamycin, rokitamycin, rolitetracycline, rosaramicin, roxithromycin, spectinomycin, spiramycin, streptomycin, sulfacetamide, sulfabenzamide, sulfadiazine, sulfadoxine, sulfamerazine, sulfamethazine, sulfamethizole, sulfamethoxazole, tobramycin, troleandomycin, salts thereof, derivatives thereof, and mixtures thereof.

15. The composition of claim 1, wherein said composition further comprises an excipient selected from the group consisting of surfactants, preservatives, emollients, humectants, fluid alkyl alcohols, thickening agents, emulsifiers, suspending agents, pH modifiers/buffering agents, chelating agents, sun filters, derivatives thereof, and mixtures thereof.

16. The composition of claim 1, wherein said composition further comprises excipients selected from the group consisting of a carbomer, a polyacrylic polymer, glycerin, sodium hydroxide, sodium thiosulfate, propyl gallate, an alkyl paraben, purified water, titanium dioxide, zinc oxide, and mixtures thereof.

17. The composition of claim 1, wherein one or more of said benzoyl peroxide, said antibiotic or a pharmaceutically acceptable salt or ester thereof, and said retinoid or a pharmaceutically acceptable salt thereof is encapsulated or entrapped in a solid or semi-solid ingredient.

18. The composition of claim 1, wherein one or more of said benzoyl peroxide, said antibiotic or a pharmaceutically acceptable salt or ester thereof, and said retinoid or a pharmaceutically acceptable salt thereof is in a solution, dispersion, or suspension in said storage-stable mixture.

19. The composition of claim 18, wherein said benzoyl peroxide and said retinoid or a pharmaceutically acceptable salt thereof are in suspension and said antibiotic or a pharmaceutically acceptable salt thereof is in solution.

20. The composition of claim 1, wherein the benzoyl peroxide has a particle size that is reduced through processing or the use of solvents.

21. The composition of claim 1, wherein a combination of any two of the benzoyl peroxide, the antibiotic or a pharmaceutically acceptable salt or ester thereof, and the retinoid or a pharmaceutically acceptable salt thereof is stable at room temperature.

22. The composition of claim 1, wherein at least two of the benzoyl peroxide, the antibiotic or a pharmaceutically acceptable salt or ester thereof, and the retinoid or a pharmaceutically acceptable salt thereof requires refrigeration.

23. A topical composition for treating a skin disorder or condition, which comprises:
a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,
wherein one or more of said benzoyl peroxide, said antibiotic or a pharmaceutically acceptable salt or ester thereof, and said retinoid or a pharmaceutically acceptable salt thereof is encapsulated or entrapped in a solid or a semi-solid ingredient, and
wherein the composition has a final pH of about 3 to about 8.

24. The composition of claim 23, wherein said solid or semi-solid ingredient has a melting point at about a mammal's body temperature.

25. The composition of claim 24, wherein said mammal is a human.

26. A topical composition for treating a skin disorder or condition, which comprises:
a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,
wherein said composition maintains a concentration of each of said benzoyl peroxide, said antibiotic or a pharmaceutically acceptable salt or ester thereof, and said retinoid or a pharmaceutically acceptable salt thereof ingredients that is at least 90% of a label claim for each of said ingredients.

27. A method for treating a skin disorder or condition in a patient comprising topically administering to a patient in need thereof a topical composition in an amount effective to treat said skin disorder, wherein said composition comprises:
a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,
wherein the composition has a final pH of about 3 to about 8, and wherein the composition has a viscosity lower than the viscosity of the benzoyl peroxide-containing composition before obtaining said storage-stable mixture.

28. The method of claim 27, wherein said skin disorder or condition includes a microbial infection.

29. The method of claim 27, wherein said skin disorder is selected from the group consisting of acne, impetigo, rosacea, psoriasis, dermatitis, secondary skin infections, responsive dermatoses, and combinations thereof.

30. The method of claim 27, wherein said topical composition is administered either concomitantly or sequentially with an additional pharmaceutical dosage form effective to treat said skin disorder or condition.

31. A topical composition for treating a skin disorder or condition, which comprises:
a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,
wherein the composition has a final pH of about 3 to about 8 that contributes to stability of said topical composition.

32. The composition of claim 31, wherein said topical composition remains storage stable under conditions selected from the group consisting of freezer conditions of less than about 0° C., about 2° C. to about 8° C., about 8° C. to about 15° C., about 23° C. to about 27° C., up to about 25° C., and about 15° C. to about 30° C.

33. A topical composition for treating a skin disorder or condition, which comprises:
a storage-stable mixture of a benzoyl peroxide-containing composition, an antibiotic or a pharmaceutically acceptable salt or ester thereof, a retinoid or a pharmaceutically acceptable salt thereof, and a pharmaceutically acceptable carrier,
wherein the composition is storage stable at a refrigerated temperature of not more than 15° C. for at least 60 days.

34. The composition of claim 33, wherein said composition is storage stable at a refrigerated temperature of about 2° C. to about 8° C. for at least 60 days.
